(19) United States
(12) Patent Application Publication
(10) Pub. No.: US 2023/0404551 A1
(43) Pub. Date: Dec. 21, 2023

(54) Deployable Tubular Biopsy Device

(71) Applicant: University of Washington, Seattle, WA (US)

(72) Inventors: Matthew Carson, Seattle, WA (US); Malay Patel, Seattle, WA (US); Eric J. Seibel, Seattle, WA (US); Lucas Meza, Seattle, WA (US)

(21) Appl. No.: 18/252,207

(22) PCT Filed: Nov. 18, 2021
(86) PCT No.: PCT/US2021/059881

§ 371 Date: May 9, 2023

Related U.S. Application Data
(60) Provisional application No. 63/115,269, filed on Nov. 18, 2020.

(51) Int. Cl. A61B 10/02; A61B 10/04

(52) U.S. Cl. CPC A61B 10/0266; A61B 10/04; A61B 2017/00986

(57) ABSTRACT

The present disclosure provides a device including a cylindrical member having a distal end and a proximal end. The cylindrical member includes a plurality of longitudinal slits positioned between the distal end and the proximal end to thereby create a plurality of strips positioned between the plurality of longitudinal slits. The device also includes an elongated hollow tube having a distal end and a proximal end. The elongated hollow tube is coupled to the proximal end of the cylindrical member. The device also includes a rod having a distal end and a proximal end.

### CROSS-REFERENCE TO RELATED APPLICATIONS

This application claims priority to U.S. Provisional Patent Application No. 63/115,269, filed Nov. 18, 2020, the contents of which are hereby incorporated by reference in their entirety.

### STATEMENT REGARDING FEDERAL FUNDING

This invention was made with government support under Grant No. R01 CA200007, awarded by the National Institutes of Health (NIH). The government has certain rights in the invention.

### BACKGROUND

Unless otherwise indicated herein, the materials described in this section are not prior art to the claims in this application and are not admitted to be prior art by inclusion in this section.

Endoscopic retrograde cholangiopancreatography (ERCP) is a broad group of medical procedures that are applied to solve problems associated with the pancreatobiliary system. A part of this procedure typically involves the procurement of a biopsy from either the pancreatic or biliary ducts. A bile duct biopsy procedure is one in which a sample of tissue or cells is safely extracted to diagnose disease, which is often a preliminary step before determining if the patient needs to have a surgical resection. The tissue sampling technique must have high sensitivity for detecting malignancy while maintaining sufficient specificity; and as with any procedure, ERCP-based sampling techniques should be safe, simple, and relatively inexpensive so they can be widely used. The remote, narrow-branched structure of the bile duct (with an inner diameter typically less than 6 mm) makes it difficult to view constrictions or maneuver the tool to perform the sampling procedure. The current standard for biopsy guided acquisition is use of fluoroscopy to direct the device to areas in question. Studies have demonstrated that biliary stricture sampling specimens acquire miniscule amounts of tissue and frequently contain insufficient cellularity, often leading to false-negative diagnoses. The most common biopsy method in the bile duct is brush sampling of cells for cytology. In this procedure, a guidewire steers a brush passed into a duct which is often constricted. When the guidewire is pulled, the epithelial cells on the duct walls get trapped in the dense bristles and in this way the sample can be obtained. However, the sensitivity of standard biliary brushings historically is low, around 30%-60%. It has been suggested that the low sensitivity of brush cytology is mainly due to inadequate cellular sampling, which may be because many malignancies compress the biliary tree from outside or because of the fibrolamellar growth of many bile duct tumors. In general, it is preferred that epithelial to deeper submucosal tissues be obtained for proper tissue diagnosis.

A less common method of sampling tissue in the biliary ducts is the forceps biopsy procedure. The forceps are a pair of sharp-edged jaws controlled by a coaxial wire within a flexible shaft. These forceps can be opened or closed by pressing and releasing the knob at the end of a shaft which is present in the hands of the operator. In operation, the two sharp edged cups of the forceps are opened, pressed against tissue in question, closed, and then the device is quickly pulled back to rip out a small piece of tissue from the bile duct wall. This technique is more time consuming and more technically challenging than brushing and thus is less commonly used. The forceps are ill-designed for small ducts because the opening process expands the tool which restricts access to the constrictions where diseases often reside. Further, the rigid and linear nature of the forceps preclude directed tissue sampling from straight luminal surfaces. For example, biopsying tissue behind an angled bile duct cannot be performed as the device cannot be angled acutely. In some patients, biopsies are not feasible as the positioning of the endoscope below the stomach does not allow for passage of the standard-sized forceps out of the accessory channel due to the thick and thus more rigid nature of the device.

Current techniques are limited in their ability to provide reassurance in the management of benign disease or targeted therapy in the face of malignancy. Due to the issues with existing procedures outlined above, there is room for improvement in reducing complexity and cost while raising sensitivity and specificity of these biopsy procedures.

### SUMMARY

The present disclosure provides a biopsy cutting tool deployed by catheter into anatomical ducts, such as the biliary duct or the coronal artery, to collect biopsy specimens from the walls of the anatomical duct.

In particular, in one aspect, a device is provided including (a) a cylindrical member having a distal end and a proximal end, wherein the cylindrical member includes a plurality of longitudinal slits positioned between the distal end and the proximal end to thereby create a plurality of strips positioned between the plurality of longitudinal slits, (b) an elongated hollow tube having a distal end and a proximal end, wherein the elongated hollow tube is coupled to the proximal end of the cylindrical member, and (c) a rod having a distal end and a proximal end, wherein the rod is positioned at least partially within the elongated hollow tube, wherein an axial movement of the rod with respect to the elongated hollow tube causes the cylindrical member to transition from a retracted position in which the plurality of strips are aligned with the distal end and the proximal end of the cylindrical member to an expanded position in which the plurality of strips protrude radially outward from the distal end and the proximal end of the cylindrical member, and wherein a diameter of the cylindrical member in the expanded position is greater than a diameter of the cylindrical member in the retracted position.

In a second aspect, a method is provided. The method may include (a) positioning the cylindrical member of the device of the first aspect adjacent the target anatomy, (b) transitioning the cylindrical member from the retracted position to the expanded position, (c) moving the cylindrical member with respect to the target anatomy to capture the biopsy sample from the target anatomy, (d) transitioning the cylindrical member from the expanded position to the retracted position, and (e) removing the device from the target anatomy.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates an example device in a target anatomy, according to an example embodiment.

FIG. 2 illustrates a side cross-sectional view of an example device in a retracted position, according to an example embodiment.

FIG. 3 illustrates a side cross-sectional view of the device of FIG. 2 in an expanded position, according to an example embodiment.

FIG. 4A illustrates a side view of a cylindrical member of the device in a retracted position, according to an example embodiment.

FIG. 4B illustrates a side view of the cylindrical member of FIG. 4A in an expanded position, according to an example embodiment.

FIG. 5 illustrates a top view of the device of FIG. 2 in an expanded position and rotational cutting motion to take a tissue sample, according to an example embodiment.

FIG. 6 illustrates a side cross-sectional view of the device of FIG. 2 in the expanded position with a tissue sample removed from the target anatomy, according to an example embodiment.

FIG. 7 illustrates a side cross-sectional view of the device of FIG. 2 in the retracted position with a tissue sample removed from the target anatomy, according to an example embodiment.

FIG. 8 illustrates a side cross-sectional view of another example device including fluid suction allowing transport of the tissue sample from the device, according to an example embodiment.

FIG. 9 illustrates a side cross-sectional view of another example device including a filter, according to an example embodiment.

FIG. 10 illustrates a side cross-sectional view of another example device including a plurality of spikes, according to an example embodiment.

FIG. 11 illustrates a side cross-sectional view of another example device including a spring, according to an example embodiment.

FIG. 12 illustrates a side view of another example device including a clamp, a hand grip, and a ratchet mechanism, according to an example embodiment.

FIG. 13 illustrates a mechanism to apply a simultaneous axial displacement and twisting force as the device transitions from the retracted position to the expanded position, according to an example embodiment.

FIG. 14 illustrates the device of FIG. 13 in the expanded position, according to an example embodiment.

FIG. 15 illustrates the device of FIG. 13 with a guidewire, according to an example embodiment.

FIG. 16 illustrates a side view of a cylindrical member of an example device, according to an example embodiment.

FIG. 17 illustrates a side view of a cylindrical member of another example device, according to an example embodiment.

FIG. 18 illustrates a side view of a cylindrical member of another example device, according to an example embodiment.

FIG. 19 illustrates an axial view of achieving different cutting edge characteristics based on machining of the cylindrical member, according to an example embodiment.

FIG. 20 illustrates a side cross-sectional view of an example device in a retracted position, according to an example embodiment.

FIG. 21 illustrates a side cross-sectional view of the device of FIG. 20 in an expanded position, according to an example embodiment.

FIG. 22 illustrates a side view of a cylindrical member and a second cylindrical member of another example device, according to an example embodiment.

FIG. 23 is a flowchart illustrating an example method for extracting a biopsy sample from a target anatomy, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the Figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

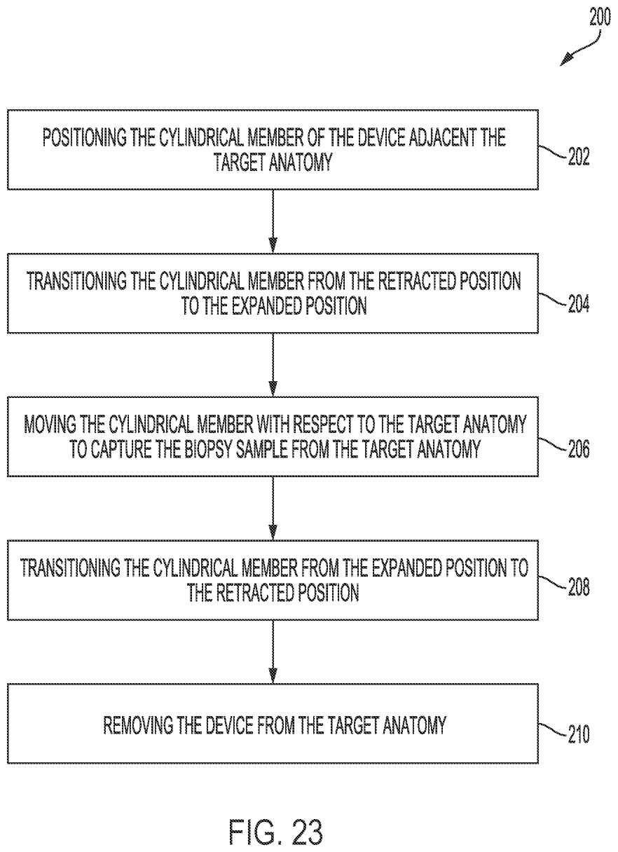

In FIG. 23, referred to above, the blocks may represent operations and/or portions thereof and lines connecting the various blocks do not imply any particular order or dependency of the operations or portions thereof. It will be understood that not all dependencies among the various disclosed operations are necessarily represented. FIG. 23 and the accompanying disclosure describing the operations of the method(s) set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

As used herein, with respect to measurements, “about” means +/−5%.

As used herein, with respect to measurements, “substantially” means +/−5%.

As used herein, the terms “biopsy sample”, “biological specimen”, “biological tissue sample”, “biopsy”, and “biospecimen” may be used interchangeably to mean a sample of biological tissue taken from a human or animal.

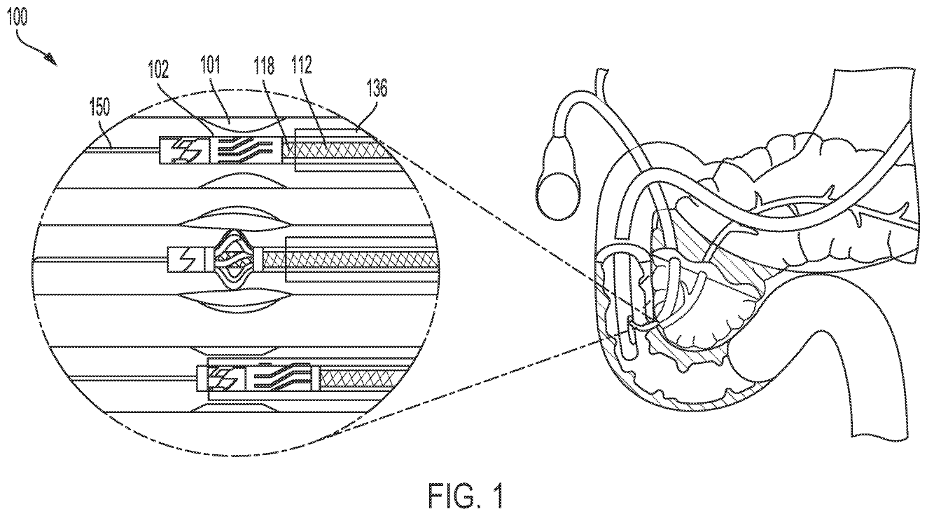

Generally, the present disclosure provides a biopsy cutting device deployed into anatomical ducts, such as the biliary duct or the coronal artery, to collect biopsy specimens from the walls of the anatomical duct. As shown in FIG. 1, the device 100 includes a cylindrical member 102. Blades or cutting edges are created in the cylindrical member 102 by cutting out a plurality of longitudinal slits 108 in the cylindrical member 102 and sharpening edges along the edges of the cylindrical member 102 left next to those cut outs. The device 100 has multiple cutting edges that are deployed outward from the longitudinal axis of the generally cylindrical member 102. Deployment of the cutting edges or blades takes place after the device 100 is placed in the target anatomy and optionally unsheathed from a sheath 136 into an anatomical duct. Once positioned in the target anatomy, an axial displacement is applied to the cylindrical member 102 via a rod 118, which conveys an axial compression and optionally a twist of the cylindrical member 102 along and about its longitudinal axis. The axial displacement causes the cutting edges to arc outward from the longitudinal axis of the device 100, thereby setting the cutting edges of the cylindrical member 102 at an increased radius from the longitudinal axis than in the previous, undeployed state. This outward radial pressure of the cylindrical member 102 against the tissue along with a displacement axially provides the cutting forces required to sample thin slices of tissue from all around the luminal surface of the target anatomy. Once a biopsy sample has been cut by the expanded cutting edges, the buckling displacement on the cylindrical member 102 is reversed, the tissue samples and fragments contained within cylindrical member 102 of the device 100 and surroundings can be captured with suction, and when the cutting tool is re-sheathed within the sheath 136.

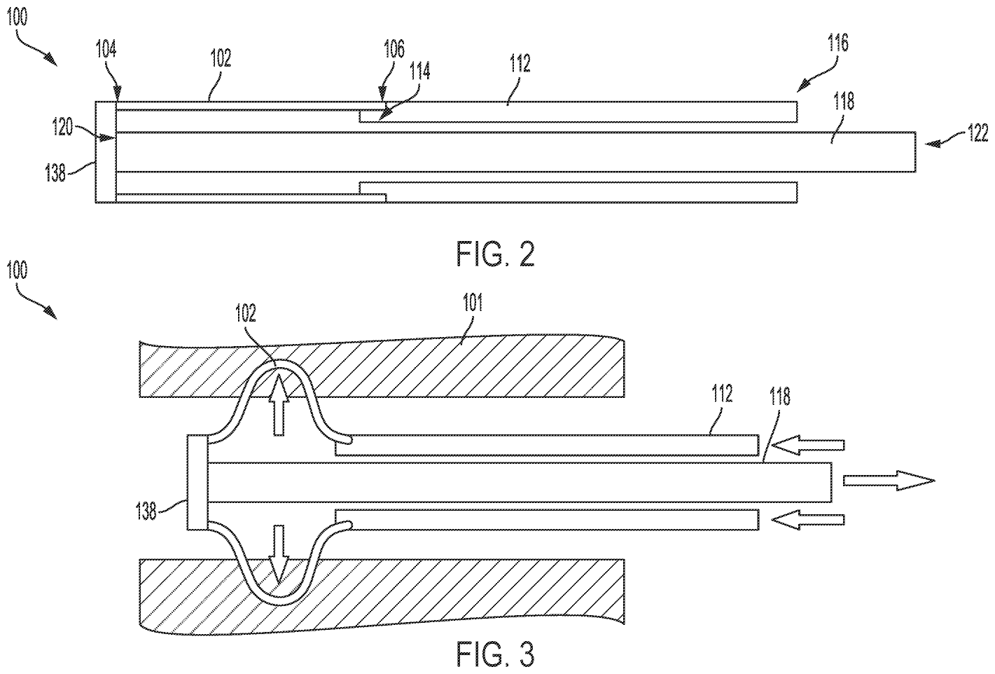

With further reference to the Figures, FIG. 2 illustrates a side cross-sectional view of an example device 100, according to an example embodiment. As shown in FIG. 2, the device 100 includes a cylindrical member 102 having a distal end 104 and a proximal end 106. The cylindrical member 102 includes a plurality of longitudinal slits 108 positioned between the distal end 104 and the proximal end 106 to thereby create a plurality of strips 110 positioned between the plurality of longitudinal slits 108. The device 100 further includes an elongated hollow tube 112 having a distal end 114 and a proximal end 116. The elongated hollow tube 112 is coupled to the proximal end 106 of the cylindrical member 102. The device 100 further includes a rod 118 having a distal end 120 and a proximal end 122. The rod 118 is positioned at least partially within the elongated hollow tube 112. An axial movement of the rod 118 with respect to the elongated hollow tube 112 causes the cylindrical member 102 to transition from a retracted position in which the plurality of strips 110 are aligned with the distal end 104 and the proximal end 106 of the cylindrical member 102 to an expanded position in which the plurality of strips 110 protrude radially outward from the distal end 104 and the proximal end 106 of the cylindrical member 102, such that a diameter of the cylindrical member 102 in the expanded position is greater than a diameter of the cylindrical member 102 in the retracted position. FIG. 2 illustrates the device 100 in the retracted position, while FIG. 3 illustrates the device 100 in the expanded position.

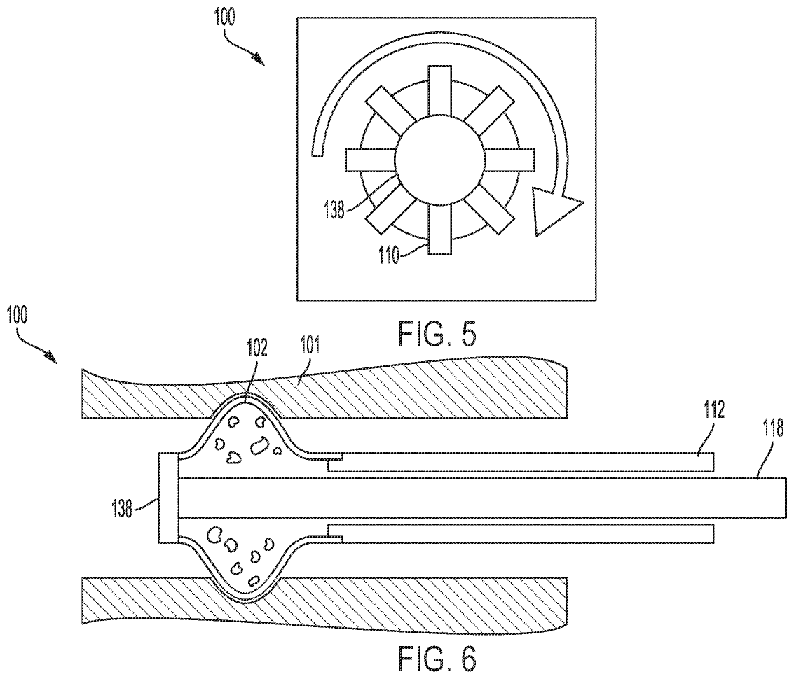

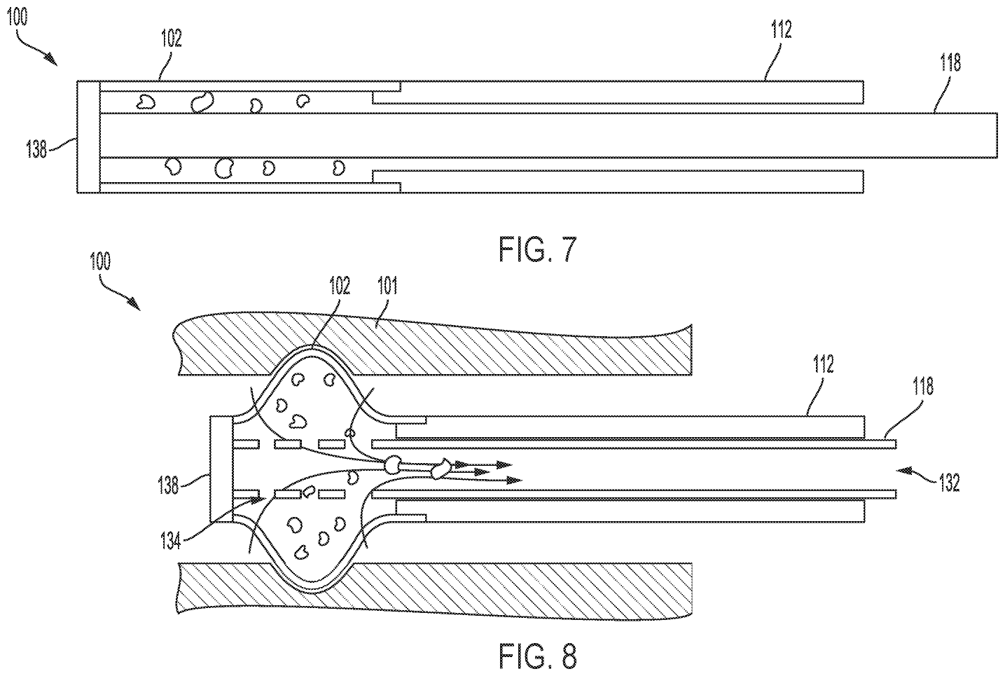

In use, by pulling on the rod 118 relative to the elongated hollow tube 112, the cylindrical member 102 is compressed, also causing the cylindrical member 102 to expand radially. The expanded cylindrical member 102 bows outward and exposes itself as a plurality of strips 110 surrounding the longitudinal axis of the cylindrical member 102, as shown in FIG. 3. The plurality of strips 110 will expand far enough outward to press into the target anatomy 101 in all directions, even if the target anatomy 101 is somewhat larger than the cylindrical member 102. The elongated hollow tube 112 and the rod 118 are together rotated from the proximal end to rotate the plurality of strips 110 through the target anatomy 101 as shown in FIG. 5, cutting out samples that will accumulate inside the plurality of strips 110, as shown in FIG. 6. The edges of the plurality of strips 110 may be rough (serrated) or sharpened to aid in the cutting, as discussed in additional detail below. After cutting, the rod 118 is released to allow the cylindrical member 102 to relax to its cylindrical form, trapping the samples for retrieval after the device 100 is removed externally, as shown in FIG. 7.

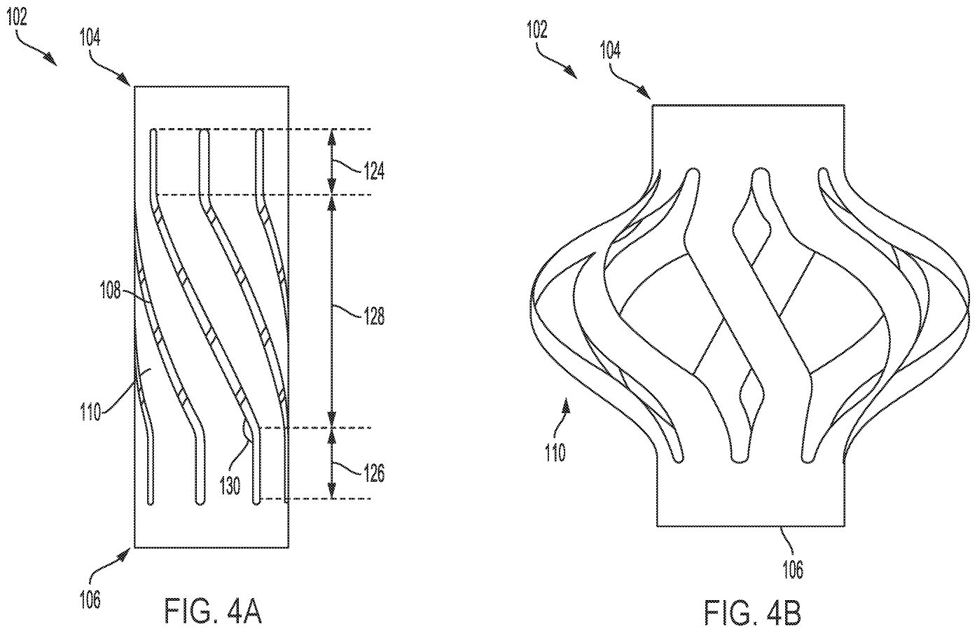

In one example, as shown in FIG. 4A, each of the plurality of longitudinal slits 108 comprise a first straight section 124, a second straight section 126, and an angled sections 128 between the first straight section 124 and the second straight section 126. In one example, an angle 130 between the first straight section 124 and each of the angled sections 128 ranges from about 105 degrees to about 135 degrees.

In one example, an outer diameter of the cylindrical member 102 in the retracted position ranges from about 0.5 mm to about 4 mm. In another example, a length of the cylindrical member 102 in the retracted position ranges from about 8 mm to about 15 mm. In one example, the diameter of the cylindrical member 102 in the expanded position ranges from about 2 greater than the diameter of the cylindrical member 102 in the retracted position to about 3 times greater than the diameter of the cylindrical member 102 in the retracted position.

The rod 118 of the device 100 may take a variety of forms. In one example, the rod 118 comprises a braided cable. In one example, the rod 118 is solid such that there is no lumen positioned therein. In another example, the rod 118 includes a lumen 132. In one such example, as shown in FIG. 8, the distal end 114 of the elongated hollow tube 112 includes a plurality of through-holes 134 to enable suction of samples extracted via the plurality of strips 110 out of the lumen 132 of the rod 118. In another example, as shown in FIG. 1, the device 100 further includes a sheath 136 configured to be removably positioned over at least a portion of the elongated hollow tube 112 and at least a portion of the cylindrical member 102. Suction is applied to the sheath 136 to thereby remove samples extracted via the plurality of strips 110 out of the sheath 136.

The cylindrical member 102 may take a variety of forms. In one example, the cylindrical member 102 comprises a shape memory material, such as Nitinol as a non-limiting example. In another example, the cylindrical member 102 comprises an elastic material that relaxes back to a cylinder form for withdrawal of the device 100 from the target anatomy. Flexibility may also be manifested in plastic in various forms in parallel with thermal or mechanical means of deployment or temporary change in the physical properties of the plastic. The plastic could be imbued with a hard material as a backbone or embedded for cutting/tearing, such as particulate diamond to enhance the cutting method (e.g., sawing). In another example, the cylindrical member 102 may be inelastic and permanently deformed when transitioned to the expanded position, then re-deformed back to a form suitable for withdrawal of the device 100 from the target anatomy such as the original cylinder or a collapsed form smaller than the original cylinder, or by a material change such as phase change or amalgam of materials. In yet another example, after expansion and cutting is performed, the cylindrical member 102 is relaxed by a heat source for example human body heat or electrical current through the cylindrical member 102.

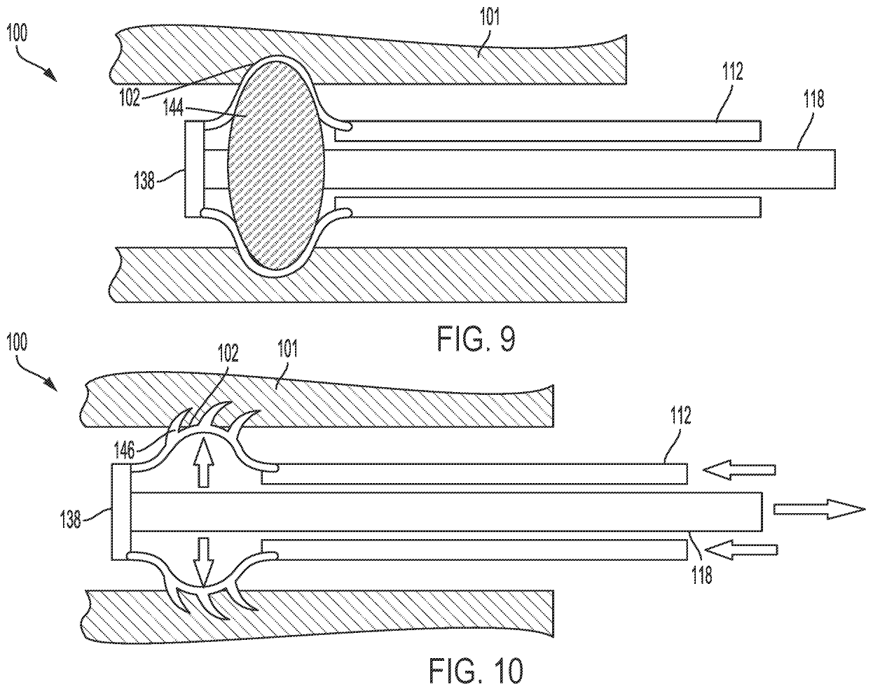

In one example, the plurality of strips 110 of the cylindrical member 102 are coupled to an electrical source to provide heat to the plurality of strips 110 to facilitate cutting of tissue at the target anatomy. In another example, as shown in FIG. 9, the device 100 further includes a filter 144 that can be used to catch cell, fragments, and other debris from the biopsy procedure either in addition or alternative to relying on capture by suction of fluid or by closure of the device. In one example, the filter 144 may be positioned distal to the device 100. In another example, the filter 144 may be positioned below the cylindrical member 102 surrounding the rod 118 to thereby form a pocket to hold biopsy samples.

In one example, instead of optimization for rotational cutting, the device 100 may be optimized for cutting in the direction of the longitudinal axis of the cylindrical member 102. In one such example, as shown in FIG. 10, the plurality of strips 110 include spikes 146 extending in a radial direction away from an outer surface of the plurality of strips 110. As shown in FIG. 10, the spikes 146 may be angled in a direction towards the proximal end 116 of the elongated hollow tube 112. As such, the spikes 146 may be used to tear the tissue from the target anatomy 101 when the device 100 is moved in the proximal direction along the longitudinal axis of the cylindrical member 102. In another example, each of the plurality of strips 110 include a single cutting edge that are oriented to cut tissue when the device 100 is pulled in the proximal direction along the longitudinal axis of the cylindrical member 102.

In another embodiment, instead of expansion of the cylindrical member 102 by compression, a balloon coupled to the rod 118. Expansion of the balloon would cause the transition of the cylindrical member 102 from the retracted position to the expanded position. In this case, the balloon would be collapsed after cutting to relax the cylindrical member 102 and trap the samples. The balloon may push against an intermediate structure that pushes out the cylindrical member 102 and not against the cylindrical member 102 directly to avoid puncture of the balloon during inflation and the corresponding transition of the cylindrical member 102 from the retracted position to the expanded position.

In one example, the device 100 includes a pre-loaded spring 148 configured to rotate the cylindrical member 102 once the cylindrical member 102 is in the expanded position. In one such example, the pre-loaded spring 148 is automatically triggered once the rod 118 exceeds a certain length of movement in a proximal direction. In another example, the pre-loaded spring 148 may be actuated manually via a button or other trigger mechanism.

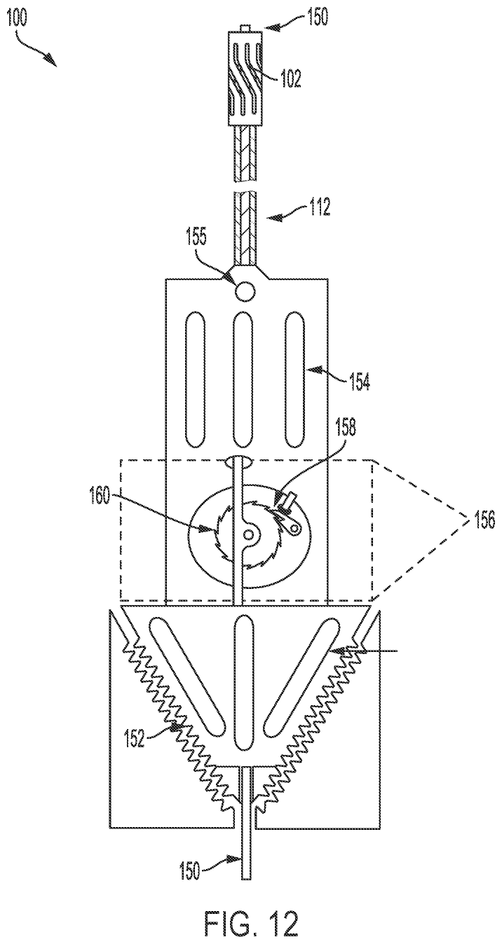

In one example, as shown in FIG. 12, the rod 118 comprises a guidewire 150. In one such example, the device 100 further includes a clamp 152 configured to prevent a longitudinal movement of the guidewire 150. The device 100 may further include a hand grip 154 coupled to the proximal end 116 of the elongated hollow tube 112. The hand grip 154 is positioned between the clamp 152 and the elongated hollow tube 112. The device 100 may further include a ratchet mechanism 156 positioned between the clamp 152 and the elongated hollow tube 112. The ratchet mechanism 156 maintains a position of the hand grip 154 with respect to the cylindrical member 102 to hold the cylindrical member 102 in the expanded position until the ratchet mechanism 156 is released.

In use, the guidewire 150 may inserted into the biliary duct or other small vessel which is extended past the region of interest for biopsy which can be user X-ray fluoroscopy or optical endoscope guidance. The cylindrical member 102 located at tip of the elongated hollow tube 112 is threaded over the guidewire 150 and through the clamp 152 of the hand grip 154. The clamp 152 may be applied to the guidewire 150 by twisting the knobs on the hand piece. The proximal end of hand grip 154 may be extended using the ratchet mechanism 156, which compresses the cylindrical member 102, thereby transitioning the cylindrical member 102 from the retracted position to the expanded position. The ratchet mechanism 156 may include a pawl 158 and a ratchet 160, as an example. The displacement of the guidewire 150 is relative to the proximal end 116 of the elongated hollow tube 112, which does not collapse under the compression with guidewire tension load. With the ratchet mechanism 156 holding the plurality of strips 110 radially outward in the expanded position, the entire hand grip 154 and elongated hollow tube 112 may be pulled to cut the tissue surrounding the cylindrical member 102. Then the ratchet mechanism 156 may be released, which releases the tension on the guidewire 150 which allows the cylindrical member 102 to relax and straighten out to roughly its original shape. In some embodiments, as discussed above, the device 100 is capable of sucking fluid from a port 155 on the hand grip 154, which can be accomplished using an empty syringe and reducing pressure by pulling back on the plunger.

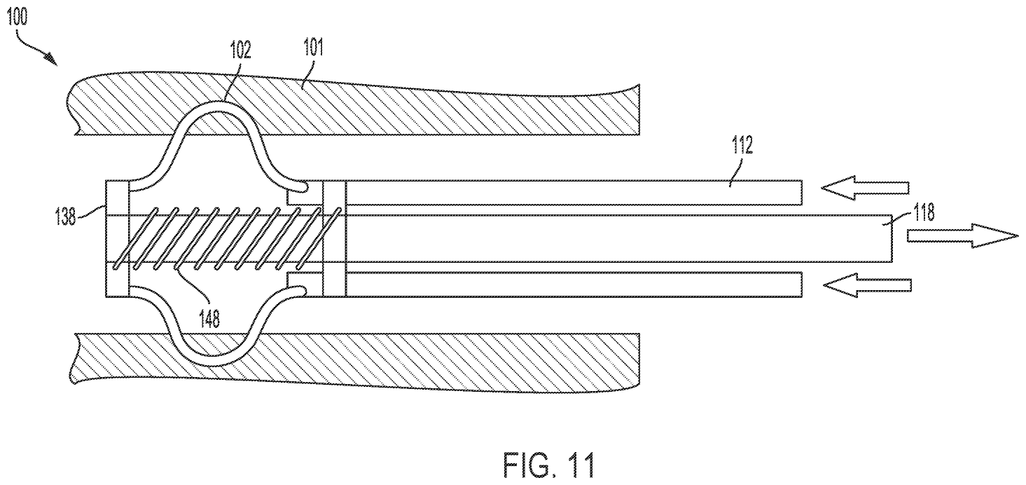

As shown in FIGS. 2-3, 5-11, and 13-15, the device 100 may include a cap 138 positioned at the distal end 104 of the cylindrical member 102, and the distal end 120 of the rod 118 is coupled to the cap 138. In one example, a diameter of the cap 138 is equal to the diameter of the cylindrical member 102 in the retracted position. The cap 138 may take a variety of forms. In one example, the cap 138 is cylindrical in shape. In another example, the cap 138 is conical in shape. As such, the cap 138 does not have to be flat and orthogonal to the pull wire axis, but instead can be shaped to fit inside a constricted lumen. In one example, the rod 118 can be a guidewire that extends through the cap 138 for a distance extending distal to the distal end 104 of the cylindrical member 102.

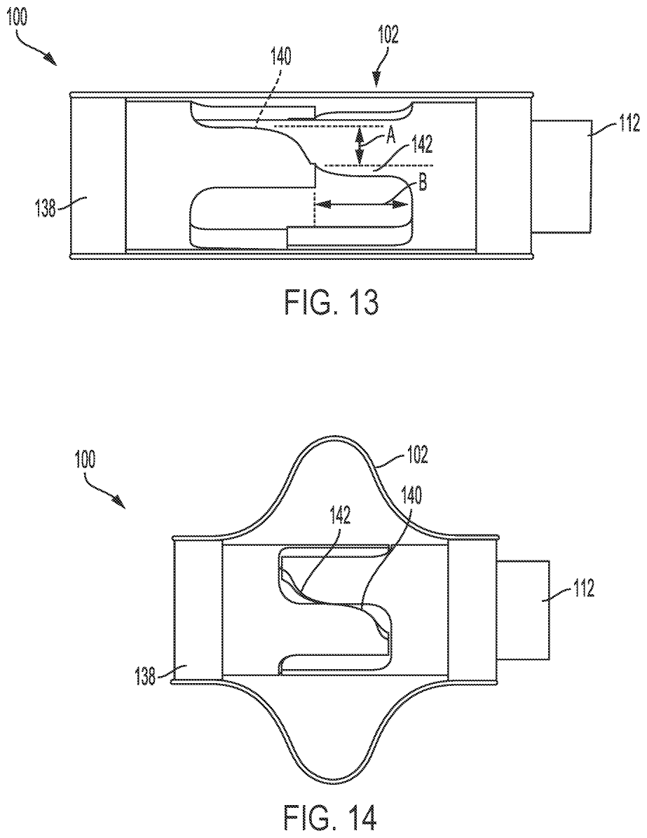

As shown in FIGS. 13-15, in one example, the cap 138 includes a first cam surface 140 and the elongated hollow tube 112 includes a second cam surface 142 such that an interaction between the first cam surface 140 and the second cam surface 142 occurs when the cylindrical member 102 transitions from the retracted position to the expanded position. The interaction between the first cam surface 140 and the second cam surface 142 causes the cylindrical member 102 to rotate about a longitudinal axis of the cylindrical member 102. Such a rotation of the cylindrical member 102 may be help to ensure a biological tissue sample is captured by the device 100. To minimize the overall work in compressing the device 100 to expand the cylindrical member 102, the first cam surface 140 and second cam surfaces 142 comprise non-linear surface that produces an amount of twist that does not have to be linearly related to the distance of axial compression. As such, the plurality of strips 110 can be controlled to the preferred bowing outward with axial compression by adding a twisting motion in parallel with the compressing motion.

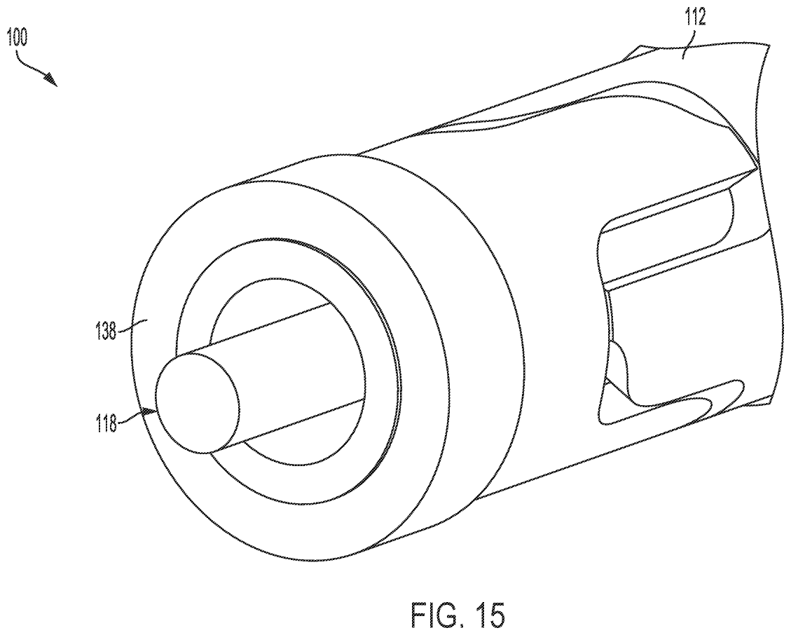

One method to implement this combined twisting-with-compression is shown in FIGS. 13-15. In this instance, the first cam surface 140 and the second cam surface 142 axially fit together and one part slides against the other. Some torque is used to maintain the contact point between the first cam surface 140 and the second cam surface 142. In this instance, the spring-force of the device 100 provides one half of the torque and whatever is attached to the proximal end of the device 100 provides the reactive torque. The range of twist is controlled by that indicated with ‘A’ in FIG. 13. The range of compression is controlled by that indicated with ‘B’ in FIG. 13. Because this device design is hollow, the central core may be accessible all the way to outside the body for the introduction of a guidewire, an inner-catheter, a working tool, or a narrow endoscope like the SFE, as shown in FIG. 15. FIG. 14 illustrates the device 100 of FIG. 13 in the expanded position, while FIG. 13 illustrates the device 100 in the retracted position.

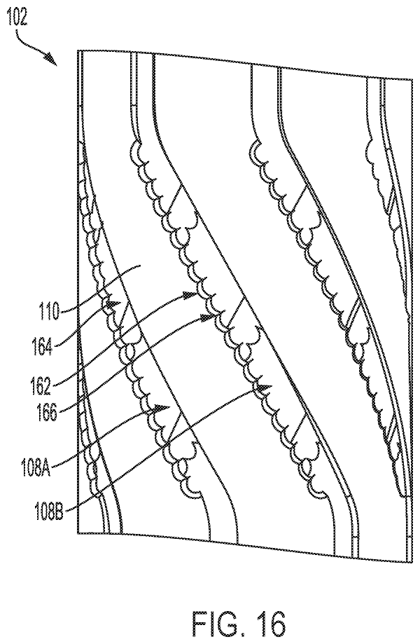

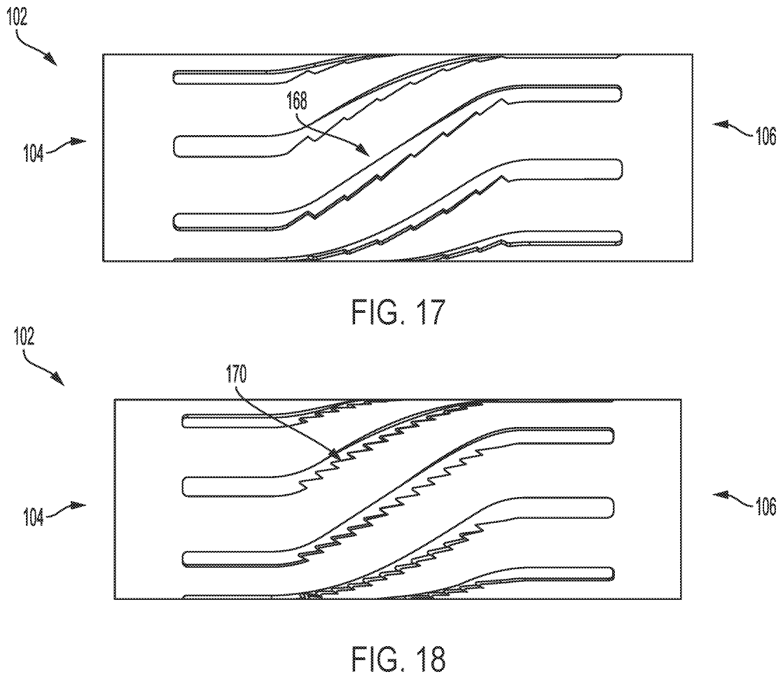

As shown in FIG. 16, each of the plurality of strips 110 includes a first edge 162 adjacent a first slit 108A of the plurality of longitudinal slits 108 and a second edge 164 adjacent a second slit 108B of the plurality of longitudinal slits 108. As shown in FIG. 16, at least the first edge 162 is a cutting edge. In one example, as shown in FIG. 16, the cutting edge is serrated 166. In another example, as shown in FIG. 17, the cutting edge comprises a plurality of teeth 168. The plurality of teeth 168 shown in FIG. 17 include points directed towards the proximal end 106 of the cylindrical member 102. In the embodiment shown in FIG. 17, both the first edge 162 and the second edge 164 including the plurality of teeth 168 are under-cut to form two sharpened edges. In another example, as shown in FIG. 18, the cutting edge comprises a plurality of barbs 170. The plurality of barbs 170 shown in FIG. 18 include points directed towards the distal end 104 of the cylindrical member 102. In one example, only the first edge 162 is a cutting edge. In another example, both the first edge 162 and the second edge 164 are cutting edges.

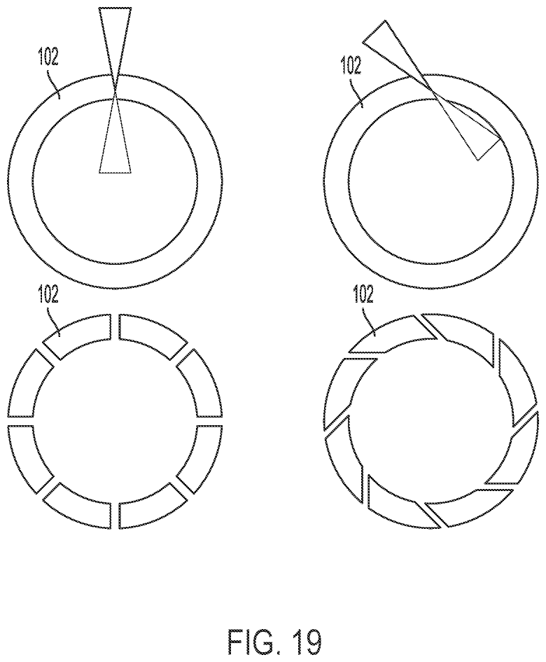

One or more of the cutting edges described above can manufactured by angling of a laser beam used for machining during the manufacturing to create angled edges. FIG. 19 illustrates the axial view of achieving different cutting edge characteristics based on machining of the cylindrical member 102. In particular, FIG. 19 illustrates both a vertical cut which creates a substantially flat edge, and an angled which creates a more sharp edge. In one example, one of the first edge 162 or the second edge 164 comprise an angled cut such that they are undercut in a direction towards the longitudinal axis of the cylindrical member 102. As shown in FIG. 19, such an undercut results in the exterior width of each of the plurality of slits 110 is greater than the interior width of each of the plurality of slits 110. In another example, both the first edge 162 and the second edge 164 comprise an angled cut such that they are undercut in a direction towards the longitudinal axis of the cylindrical member 102.

Alternative means for manufacturing other than laser machining is by adding roughness and pitting from electrical discharge machining (EDM). Other manufacturing techniques are possible as well. The cutting edges of the device 100 described above may be sharpened by selective material removal or treated in various ways to enhance the removal of tissue. Some varieties of edge treatment could be similar to the method described below for manufacturing, but not necessarily the same (make the device one way, sharpen it with another). The cutting edges may be enhanced by the addition of a material or application of a process that increases adhesion to the tissue to tear instead of cutting. Enhanced cutting may be addressed by the addition of diamond or other hard particulate matter, possibly in a matrix-base on the plastic or in the plastic itself to provide a sawing action instead with or instead of a directly penetrating force.

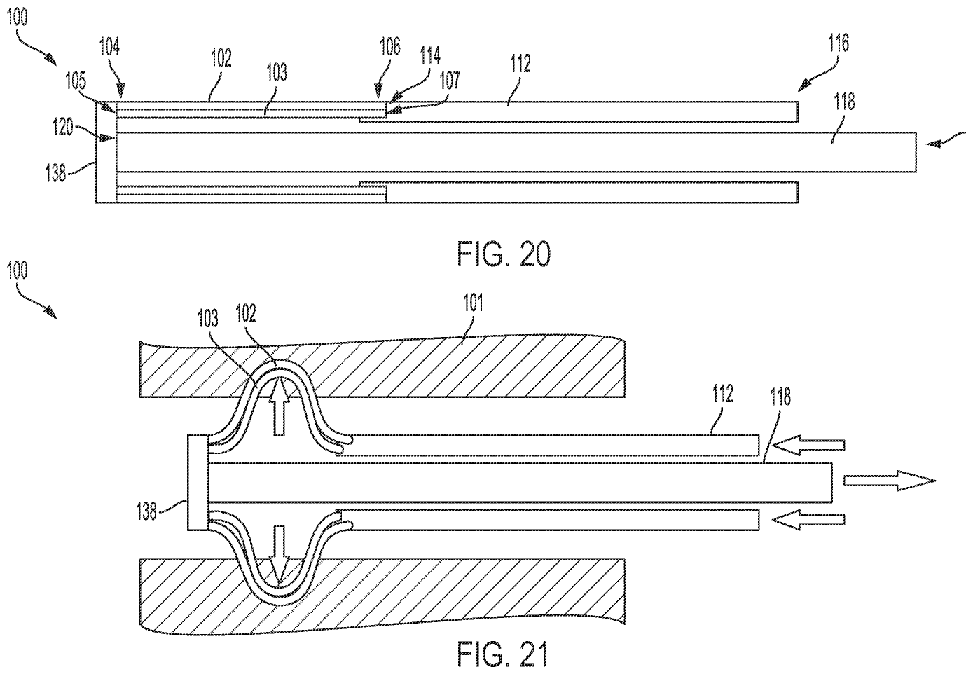

In another example, as shown in FIG. 20, the device 100 may include a second cylindrical member 103 having a distal end 105 and a proximal end 107. The second cylindrical member 103 may be positioned inside of the cylindrical member 102. The second cylindrical member 103 includes a second plurality of longitudinal slits 109 positioned between the distal end 105 and the proximal end 107 to thereby create a plurality of strips 111 positioned between the plurality of longitudinal slits 109. The elongated hollow tube 112 is coupled to the proximal end 107 of the second cylindrical member 103. Similar to cylindrical member 102, an axial movement of the rod 118 with respect to the elongated hollow tube 112 causes the second cylindrical member 103 to transition from a retracted position (shown in FIG. 20) in which the second plurality of strips 111 are aligned with the distal end 105 and the proximal end 107 of the second cylindrical member 103 to an expanded position (shown in FIG. 21) in which the second plurality of strips 111 protrude radially outward from the distal end 105 and the proximal end 107 of the second cylindrical member 103. A diameter of the second cylindrical member 103 in the expanded position is greater than a diameter of the second cylindrical member 103 in the retracted position.

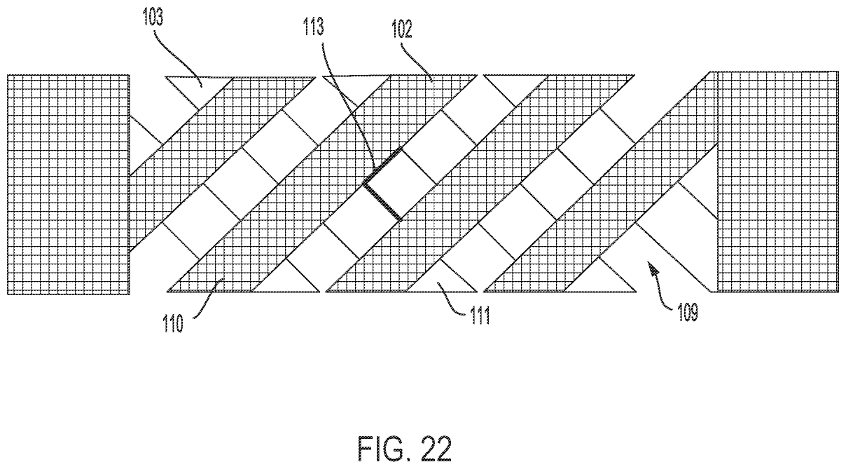

In such an example, as shown in FIGS. 20-21, the cylindrical member 102 acts as an outer tube, and the second cylindrical member 103 acts as an inner tube. The cylindrical member 102 and the second cylindrical member 103 are nested tubes that can be expanded radially by axial compression applied by the rod 118, as described above. In one particular example, as shown in FIG. 22, the cylindrical member 102 and the second cylindrical member 103 include opposing spiral bands that act in tandem to present a cutting array that would not be possible with a single cylindrical member. In particular, as shown in FIG. 22, the overlapping edges each of the plurality of strips 110 with the second plurality of strips 111 create a focused cutting region 113. In one example, the cylindrical member 102 and the second cylindrical member 103 are rotated in tandem when they are both in the expanded position. In another example, the cylindrical member 102 and the second cylindrical member 103 are rotated independently when they are both in the expanded position. Such an arrangement may provide improved tissue entrapment or cutting ability. Other arrangements of the second cylindrical member 103 are possible as well.

In use, the plurality of strips 110 are manipulated for cutting by either rotation around and/or movement along the axis of the cylinder. Because of the axially-symmetric expansion of the blades pushing against the entire surface of the surrounding luminal walls, the difficulties of obtaining correct blade pressure and aiming to the sampling site with a narrow flexible tool within a viscoelastic duct tissue are mitigated. Cutting tissue under tension can be done more easily with this radial pressure, and the cells and tissue can press through the open slots between the blades. The displacement of the plurality of strips 110 can more easily cut when the cutting edges of the plurality of strips 110 are serrated, as discussed above.

In use, as shown in FIG. 1, a sheath 136 having a device 100 near the distal end is introduced through the working channel of a macro-sized endoscope (motherscope) to a distal collection site in the human body. The sheath 136 is micro-sized to collect biopsies from previously inaccessible locations in the human body such as the constructed bile duct or peripheral lung airway. The micro-sized sheath 136 may or may not have imaging capabilities (babyscope). The device 100 has the novel feature of allowing tissue fragments and slices of tissue to be sampled from luminal walls without lateral steering. The geometry of the cylindrical member 102 of the device includes various patterns of slits, cutouts, or variations in thickness that determine the geometry of the cutting shapes and arrangement. The unexpanded cylindrical member 102 has a smooth outer surface during introduction to the sampling location. When at the biopsy location, such as a constriction in the duct, the cylindrical member 102 is expanded to form a circular array of sharp parts that can be used for cutting or tearing. The sharp parts can be expanded, then used to cut and/or tear tissue that it contacts with a secondary motion, such as releasing a rotational twisting motion. Alternatively, the expansion and cutting and/or tearing motion can be in the same motion, such as pulling down on a guidewire to compress the device which expands out the blades while also pulling through the luminal constriction. Ideally a sheet of epithelial tissue is sampled by the separated blades like a cheese slicer across the entire luminal surface of interest, which is then captured by the cylindrical member 102 as the cylindrical member 102 transitions back from the expanded position to the retracted position. To integrate with optical imaging, in one example a scanning fiber endoscope (SFE) may act as the guidewire that can be pulled to axially compress and expand the diameter and cylindrical member 102 of the device 100. Alternatively, a standard metal guidewire can be used as shown in cross-section coming out of the integrated twist-compress guidance device in FIG. 15. A third alternative is having both the ultrathin endoscope (<1 mm diameter) and the guidewire (<1 mm diameter) side-by-side within the device 100. Other alternatives are possible as well.

Manufacturing of various components of the device 100 may be injection molded or cast in high volumes, while various components of the device 100 may be 3D-printing in small volume manufacturing. The materials can be lubricious smooth plastic surfaces (PTFE, Teflon, or Delrin, as non-limiting examples). Various components of the device 100 may comprise the same materials, or they may comprise different materials.

The plurality of strips 110 can be molded in place (by either deformation or some form of casting), embossed or punched, etched by various means including chemical, electrical, plasma, or other volumetric fields. Another approach is the use of directed-energy sources such as lasers, or high-energy particle beams. The energy source for the etching may also be a volumetric field or flux applied to a mask on the material to change the material properties that result in non-uniform properties across the body of the device. The plurality of strips 110 can also be milled by mechanical means such as cutting, or grinding. Any of the above may be combined with another process during some phase of the construction. The entire cylindrical member 102 may also be built up by layers, either concentrically accumulated from cylindrical forms, or by layer with various methods including vapor deposition or other chemical means such as plating for example in a solution.

FIG. 23 is a block diagram of an example method for extracting a biopsy sample from a target anatomy. Method 200 shown in FIG. 23 presents an embodiment of a method that could be used by the device 100 as described in FIGS. 1-22, as examples. Method 200 may include one or more operations, functions, or actions as illustrated by one or more of blocks 202-210. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In addition, for the method 200 and other processes and methods disclosed herein, the block diagram shows functionality and operation of one possible implementation of present embodiments. In this regard, each block may represent a module, a segment, or a portion of program code, which includes one or more instructions executable by a processor or computing device for implementing specific logical functions or steps in the process. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

Initially, at block 202, the method 200 includes positioning the cylindrical member 102 of the device 100 described above adjacent the target anatomy 101. At block 204, the method 200 includes transitioning the cylindrical member 102 from the retracted position to the expanded position. At block 206, the method 200 includes moving the cylindrical member 102 with respect to the target anatomy 101 to capture the biopsy sample from the target anatomy 101. At block 208, the method 200 includes transitioning the cylindrical member 102 from the expanded position to the retracted position. At block 210, the method 200 includes removing the device 100 from the target anatomy. In one example, the target anatomy 101 comprises a bile duct.

In one example, the step of moving the cylindrical member 102 with respect to the target anatomy 101 to capture the biopsy sample from the target anatomy 101 comprises rotating the cylindrical member 102 about a longitudinal axis of the cylindrical member 102. In another example, the step of moving the cylindrical member 102 with respect to the target anatomy 101 to capture the biopsy sample from the target anatomy 101 comprises moving the cylindrical member 102 in a proximal direction with respect to the target anatomy 101. In another example, the step of moving the cylindrical member 102 with respect to the target anatomy 101 to capture the biopsy sample from the target anatomy 101 comprises simultaneously rotating the cylindrical member 102 about a longitudinal axis of the cylindrical member 102 and moving the cylindrical member 102 in a proximal direction with respect to the target anatomy 101.

In one example, the method 200 further includes (i) positioning a guidewire 150 adjacent the target anatomy, and (ii) loading the device 100 onto the guidewire 150 to thereby position the cylindrical member 102 of the device 100 adjacent the target anatomy 101.

In yet another example, the method 200 further includes applying suction to a sheath 136 that is removably positioned over at least a portion of the elongated hollow tube 112 and at least a portion of the cylindrical member 102 to thereby remove the biopsy sample from the cylindrical member 102.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

1. A device comprising:
a cylindrical member having a distal end and a proximal end, wherein the cylindrical member includes a plurality of longitudinal slits positioned between the distal end and the proximal end to thereby create a plurality of strips positioned between the plurality of longitudinal slits;
an elongated hollow tube having a distal end and a proximal end, wherein the elongated hollow tube is coupled to the proximal end of the cylindrical member; and
a rod having a distal end and a proximal end, wherein the rod is positioned at least partially within the elongated hollow tube, wherein an axial movement of the rod with respect to the elongated hollow tube causes the cylindrical member to transition from a retracted position in which the plurality of strips are aligned with the distal end and the proximal end of the cylindrical member to an expanded position in which the plurality of strips protrude radially outward from the distal end and the proximal end of the cylindrical member, and wherein a diameter of the cylindrical member in the expanded position is greater than a diameter of the cylindrical member in the retracted position.

2. The device of claim 1, wherein each of the plurality of longitudinal slits comprise a first straight section, a second straight section, and an angled section between the first straight section and the second straight section.

3. The device of claim 2, wherein an angle between the first straight section and the angled section ranges from about 105 degrees to about 135 degrees.

4. The device of claim 1, wherein an outer diameter of the cylindrical member ranges from about 0.5 mm to about 4 mm.

5. The device of claim 1, wherein a length of the cylindrical member ranges from about 8 mm to about 15 mm.

6. The device of claim 1, wherein the rod comprises a braided cable.

7. The device of claim 1, wherein the rod is solid.

8. The device of claim 1, wherein the rod includes a lumen, and wherein the distal end of the elongated hollow tube includes a plurality of through-holes to enable suction of samples extracted via the plurality of strips out of the lumen of the rod.

9. The device of claim 1, further comprising:
a cap positioned at the distal end of the cylindrical member, wherein the distal end of the rod is coupled to the cap.

10. The device of claim 9, wherein the cap includes a first cam surface, and wherein the elongated hollow tube includes a second cam surface, wherein an interaction between the first cam surface and the second cam surface occurs when the cylindrical member transitions from the retracted position to the expanded position, and wherein the interaction between the first cam surface and the second cam surface causes the cylindrical member to rotate about a longitudinal axis of the cylindrical member.

11. The device of claim 9, wherein a diameter of the cap is equal to the diameter of the cylindrical member in the retracted position.

12. The device of claim 9, wherein the cap is cylindrical in shape.

13. The device of claim 9, wherein the cap is conical in shape.

14. The device of claim 1, wherein the diameter of the cylindrical member in the expanded position ranges from about 1.25 times greater than the diameter of the cylindrical member in the retracted position to about 3 times greater than the diameter of the cylindrical member in the retracted position.

15. The device of claim 1, wherein the cylindrical member comprises a shape memory material.

16. The device of claim 1, wherein the rod comprises a guidewire, and wherein the device further includes:
a clamp configured to prevent a longitudinal movement of the guidewire; and
a hand grip coupled to the proximal end of the elongated hollow tube, wherein the hand grip is positioned between the clamp and the elongated hollow tube.

17. The device of claim 16, further comprising:
a ratchet mechanism positioned between the clamp and the elongated hollow tube, wherein the ratchet mechanism maintains a position of the hand grip with respect to the cylindrical member to hold the cylindrical member in the expanded position until the ratchet mechanism is released.

18. The device of claim 1, wherein the plurality of strips are coupled to an electrical source to provide heat to the plurality of strips.

19. The device of claim 1, wherein the plurality of strips include spikes extending in a radial direction away from an outer surface of the plurality of strips.

20. The device of claim 1, further comprising:
a pre-loaded spring configured to rotate the cylindrical member once the cylindrical member is in the expanded position.

21. The device of claim 20, wherein the pre-loaded spring is automatically triggered once the rod exceeds a certain length of movement in a proximal direction.

22. The device of claim 1, further comprising:
a sheath configured to be removably positioned over at least a portion of the elongated hollow tube and at least a portion of the cylindrical member.

23. The device of claim 1, wherein each of the plurality of strips includes a first edge adjacent a first slit of the plurality of longitudinal slits and a second edge adjacent a second slit of the plurality of longitudinal slits, and wherein at least the first edge is a cutting edge.

24. The device of claim 23, wherein the cutting edge is serrated.

25. The device of claim 23, wherein the cutting edge comprises a plurality of teeth.

26. The device of claim 23, wherein the cutting edge comprises a plurality of barbs.

27. The device of claim 23, wherein both the first edge and the second edge are cutting edges.

28. A method for extracting a biopsy sample from a target anatomy, the method comprising:
positioning the cylindrical member of the device of any one of claims 1-27 adjacent the target anatomy;
transitioning the cylindrical member from the retracted position to the expanded position;
moving the cylindrical member with respect to the target anatomy to capture the biopsy sample from the target anatomy;
transitioning the cylindrical member from the expanded position to the retracted position; and
removing the device from the target anatomy.

29. The method of claim 28, wherein moving the cylindrical member with respect to the target anatomy to capture the biopsy sample from the target anatomy comprises rotating the cylindrical member about a longitudinal axis of the cylindrical member.

30. The method of claim 28, wherein moving the cylindrical member with respect to the target anatomy to capture the biopsy sample from the target anatomy comprises moving the cylindrical member in a proximal direction with respect to the target anatomy.

31. The method of claim 28, wherein moving the cylindrical member with respect to the target anatomy to capture the biopsy sample from the target anatomy comprises simultaneously rotating the cylindrical member about a longitudinal axis of the cylindrical member and moving the cylindrical member in a proximal direction with respect to the target anatomy.

32. The method of claim 28, further comprising:
positioning a guidewire adjacent the target anatomy; and
loading the device onto the guidewire to thereby position the cylindrical member of the device adjacent the target anatomy.

33. The method of claim 28, further comprising:
applying suction to a sheath that is removably positioned over at least a portion of the elongated hollow tube and at least a portion of the cylindrical member to thereby remove the biopsy sample from the cylindrical member.

34. The method of claim 28, wherein the target anatomy comprises a bile duct.
